(19) United States
(12) Patent Application Publication
(10) Pub. No.: US 2009/0082636 A1
(43) Pub. Date: Mar. 26, 2009

(54) AUTOMATED CORRELATIONAL HEALTH DIAGNOSIS

(76) Inventor: Anthony Vallone, Canton, OH (US)

Correspondence Address: David J. Stern, Esq.;Eschweiler & Associates , LLC, National City Bank Building, 629 Euclid Avenue, Suite 1000, Cleveland, OH, 44114, US

(21) Appl. No.: 11/858,764

(22) Filed: Sep. 20, 2007

(51) Int. Cl. A61B 5/00

(52) U.S. Cl. 600/300

(57) ABSTRACT

Correlational health diagnosis techniques are disclosed for diagnosing a health state of an individual based on one or more health indicators, including systems and methods related thereto. The techniques involve a diagnostic database comprising at least one correlationally weighted relationship between a health indicator and a health diagnosis. The technique involves collecting at least one health indicator pertaining to the health-state of the individual, and the health indicators may be displayed as icons of predominantly pictorial form. Once at least one health indicator has been collected, the techniques involve summing the correlational weights of each health diagnosis in the diagnostic database for each collected health indicator, and selecting and outputting at least one health diagnosis having the greatest aggregate diagnostic weight. The techniques may also involve selecting one or more health treatments related to each selected health diagnosis. Each selected health diagnosis and selected health treatment may also be outputted as an icon of predominantly pictorial form for easy recognition in a standardized, language-neutral manner.

### FIELD

This disclosure relates generally to the field of health diagnosis, and more particularly to the automation of health diagnosis based on correlations with health indicators.

### BACKGROUND

This disclosure relates to the automated health diagnosis and plan of action (PoA) for an individual (such as a patient in a healthcare facility) based on the health indicators of the individual. It will be appreciated that in the present state of healthcare, many pieces of information are identified and recorded about the health state of the individual that may be diagnostically relevant. This information may derive from many sources, including a variety of individuals involved in the provision of healthcare to the individual and/or machines and devices that detect and record data pertinent to the health state of the individual. Because the collected data may be voluminous, and because the formatting of information may be inconsistent in many aspects, deductively determining the likely health diagnosis of the individual may be difficult.

### SUMMARY

The following presents a simplified summary of the disclosure in order to provide a basic understanding of some aspects of the disclosure. This summary is not an extensive overview of the disclosure. It is intended neither to identify key or critical elements of the disclosure nor to delineate the scope of the disclosure. Rather, its primary purpose is merely to present one or more concepts of the disclosure in a simplified form as a prelude to the more detailed description that is presented later.

This disclosure relates to an automated, correlational health diagnosis technique, and method and system embodiments thereof. The health indicators of the individual may be collected and recorded according to a health indicator set, which comprises a plurality of health indicators that represent pertinent indications of the health state of the individual (e.g., presenting signs and symptoms that might include sweating, dizziness, and confusion), especially in light of the individual's medical history. The health indicators are then computationally evaluated in light of the correlation of each collected health indicator with a set of health diagnoses (e.g., diabetic hypoglycemic episode, myocardial infarction [MI], and cerebrovascular accident [CVA].) Each collected health indicator that pertains to the health state of the individual is correlationally related with each health diagnosis in the set of health diagnoses, and the one or more health diagnoses that are most highly correlated to the collected health indicators may be selected and presented as diagnostic output. Automated treatments or “critical” pathways may also be provided as output, along with an expected result that may be achieved in a specified duration of time. However, alternative health diagnoses are not ruled out, and may be undertaken at the discretion of the health care professional. For example, the healthcare provider may relate the results of the automated health diagnosis techniques with standards set by the U.S. Department of Health and Human Services (CMS) and other insurance providers for acceptable probability deviances.

To the accomplishment of the foregoing and related ends, the following description and annexed drawings set forth in detail certain illustrative aspects and implementations of the disclosure. These are indicative of but a few of the various ways in which one or more aspects of this disclosure may be employed. Other aspects, advantages, and novel features of the disclosure will become apparent from the following detailed description of the disclosure when considered in conjunction with the annexed drawings.

### BRIEF DESCRIPTION OF THE DRAWINGS

FIG. 1 is an illustration of a portion of an exemplary icon set that may be used with a health diagnosis system;

FIG. 2 is an Entity-Relationship Diagram (ERD) illustration of a portion of an exemplary schema for a diagnostic database;

FIG. 3 is an illustration of a portion of an exemplary diagnostic database;

FIG. 4 is a flowchart illustration of an exemplary health diagnosis method;

FIG. 5 is an illustration of an exemplary use of the exemplary diagnostic database of FIG. 4;

FIG. 6 is a component diagram of an exemplary health diagnosis system; and

FIG. 7 is an illustration of an exemplary computer-readable medium comprising processor-executable instructions configured to perform a method in accordance with this disclosure.

### DEFINITIONS

The following terms used herein are suggested to have the following meanings. This is not intended as an exhaustive list of defined terms, but only as an interpretive aide for facilitating the reading and comprehension of the disclosure described herein. The definitions provided herein are intended to be coupled with the other sources of interpretive guidance for these terms, such as context, common usage in the field of art, and ordinary usage in the English language.

“Healthcare” and “healthcare service” are suggested to mean a service provided to a recipient of healthcare that relates to the health, functionality, and/or physical and/or mental well-being of the recipient. Such services may derive from one or several of the myriad recognized fields of healthcare, including, for instance, allopathic medicine, osteopathic medicine, physical and/or occupational therapy, dentistry, chiropractic medicine, hospice or home healthcare, and pharmaceuticals.

“Caregiver” is suggested to mean a provider of a healthcare service to an individual. “Caregiver” may include any type of healthcare practitioner, including, for instance, a physician, nurse, physical or occupational therapist, chiropractor, dentist, or home healthcare worker. The caregiver may also be a non-professional individual who is involved in providing a healthcare service to the individual, such as a relative, friend, or cohabitant. In a broader context, “caregiver” may include other parties involved in the dispensing of medication to an individual, such as a pharmacist, drug manufacturer, or insurance agent. In the case of self-administered healthcare, the caregiver may be the individual. “Caregiver” may also refer collectively to a plurality or team of such providers, either working together (e.g., a physician and a nurse) or separately (e.g., a physician and a pharmacist.)

“Computer data signal embodied in a carrier wave” is suggested to mean a carrier wave encoding computer-usable data and transmitted to a computer via a carrier wave. The carrier wave may comprise a communications transmission in a communications medium, which may be optical, sonic, electronic, magnetic, etc. At a higher level, the communications medium may comprise (for instance) a computer network, such as the internet; a cellular network; a data connection inside a computer, such as a ribbon cable that carries data in electronic form; a magnetic signal generated from a magnetic platter of a hard disk drive that stores the instructions; or an optical signal generated by reflecting a light source, such as a laser, off of an optical platter, such as a CD-ROM, that produces variable reflectivity representing information corresponding to the stored data. The data might comprise, for instance, audio data that the computer may use to produce specific sounds; it may comprise visual data that the computer may use to produce specific pictures or movies; etc. Additionally or alternatively, the data might comprise a set of computer instructions for achieving a useful result. The computer to which the computer data signal is transmitted may be configured to execute the instructions and perform the method; it may transform the instructions into a different form of processor-executable instructions (e.g., by receiving source code from the carrier signal and compiling it into a machine-executable binary); it may transmit the instructions to a second computer in order to configure the second computer to execute the instructions; it may store the instructions for later use by the same or another computer; etc. The details are not important, so long as the carrier wave encodes computer-usable data and may be transmitted to a computer via a carrier wave.

“Processor-executable instruction” is suggested to mean an instruction selected from an instruction set that is able to operate a computer processor to achieve a desired functional result. The desired functional result may be simple, such as the storage of a value in memory, or complex, such as an invocation of an advanced programming interface (API) call that produces sophisticated functionality. The instruction set may be any suitable processor-executable instruction set, including (without limitation) a native machine architecture language, machine language, Java, JavaScript, BASIC, Visual BASIC, C, C++, C#, FORTRAN, Perl, a command shell scripting language, etc. The instruction set may be declarative, such as HTML; imperative, such as C; a hybrid language; another type of instruction set; etc. The instruction set may be fully compiled, such as into a machine-executable binary; partially compiled into an intermediate language that is subsequently fully compiled; interpreted from text, etc. The instruction may be executed natively on a processor; through a parser, advanced programming interface (API), or in a virtual machine; within another application or process, such as a web browser; etc. The details are not important, so long as the instruction is able to operate a computer to achieve the desired functional result.

“Computer-readable medium” is suggested to mean a computer-operable component capable of transmitting stored data to a computer. The medium may be comparatively static, such as a solid-state storage device, or comparatively volatile, such as system RAM. The medium may be a set of hardware components, such as one or more registers or capacitors; may comprise a fixed medium, such as a platter in a hard disk drive; may comprise a removable medium, such as a CD-ROM in a CD-ROM drive; etc. The medium may be read-only; write-only; both readable and writable; etc. The medium may be read-once; read-many; write-once; write-many; etc. The medium may be accessible in any suitable fashion, such as randomly; sequentially; either randomly or sequentially; etc. The medium may be dedicated to a particular computer or device; may be simultaneously connected to and shared by multiple computers or devices; may be shared over a network; etc. The medium may store the data in any electronic medium, such as electronic, magnetic, optical, print, etc. The medium may be used to store data for a single task or application; may be used to store data for many tasks and applications; etc. The implementation of the medium is not important, so long as the medium is computer-operable and capable of transmitting stored data to a computer.

“Data representation” is suggested to mean data stored in a memory and/or computer-readable medium that represents or describes, in whole or in part, a concept or object. The concept or object so represented may be a tangible item, such as a person; or an abstract concept, such as a specific mathematical value; or a computer-operable object, such as an image. “Data representation” is particularly suggested to mean image data, which a computer may use to render a specific image. “Data representation” is also particularly suggested to mean data that represents health information, such as a health state of a hypothetical or actual individual, an aspect of a health state of a hypothetical or actual individual, etc. “Data representation” is also particularly suggested to mean data that represents a classification scheme, such as the Outcome and Assessments Information Set.

“Health condition” is suggested to mean a general category of physical conditions that may pertain to individuals described by a health information classification scheme. A health condition might describe a physical ability, such as the ability to ambulate or to perform hygiene functions; a mental ability, such as localized awareness or the ability to recall facts; a physical trait, such as the presence and condition of a wound; etc. The health condition may contain descriptors that provide options for describing the category with greater detail. For instance, the health condition may include a set of health condition descriptors, a set of health condition contexts, and a set of health condition ratings, each of which defines options for the health condition that may together describe the health condition for an individual. As one example, a health condition may represent ambulatory capabilities, and may contain a set of health condition descriptors that describe different forms of ambulation (without an assistance device, with a cane, with a rolling walker, etc.); a set of health condition contexts that describe different environments for walking (on indoor tile, on indoor carpeting, or on an outdoors uneven surface); and a set of health condition ratings that describe the wellness of an individual for this health condition (with 100% activity, with cuing and standby, with 50% activity, etc.)

“Health diagnosis” is suggested to mean a medical condition that has been determined to be descriptive of the health state of the individual based on the health indicators of the individual. Examples of health diagnoses are diseases (e.g., a type of cancer), health disorders (e.g., myocardial infarction), and impaired health conditions (e.g., poor vision.) A health diagnosis may also be positive (e.g., very good vision), neutral (e.g., adequate cognitive function), and/or incomplete (e.g., idiopathic liver dysfunction.)

“Health indicator” is suggested to mean an observed, measured, and/or reported aspect of a health condition of an individual.

“Health information classification scheme” is suggested to mean an information system for organizing and representing the health information of an individual. One such health information classification scheme is the Outcome and Assessment Information Set (OASIS) classification scheme provided by the U.S. Department of Health and Human Services.

“Health state” is suggested to mean the state of health of an individual. The health state may be the actual state of an individual's health during a time point, such as during a particular moment or day. The health state may also be a past health state, such as a health state recorded as part of the individual's medical history, either at a time point or in the individual's unspecified or protracted past. The health state may also be a projected future health state of the individual, such as a future health state that a health regimen may be able to achieve.

“Health treatment” is suggested to mean an action that may be taken by a caregiver and/or the individual in response to a health diagnosis of the individual. Such actions may include, e.g., one or more further diagnostic instructions (e.g., test hemoglobin count); one or more medical or surgical instructions (e.g., remove appendix); one or more pharmacological prescriptions (e.g., administer antibiotics); one or more nursing instructions (e.g., monitor: blood pressure for 48 hours); and/or one or more physical or occupational therapy instructions (e.g., change wound dressing every day.)

“Icon” is suggested to mean a graphic symbol and/or word whose visual form represents and/or suggests a concept.

“Individual” is suggested to mean a recipient of healthcare service provided by a caregiver. In the case of self-administered healthcare, the individual may also be the caregiver.

“Memory” is suggested to mean a computer-operable component capable of storing and/or retrieving data. The memory may be comparatively static, such as a solid-state storage device, or comparatively volatile, such as system RAM. The memory medium may be a set of hardware components, such as one or more registers or capacitors; may utilize a fixed medium, such as a platter in a hard disk drive; may utilize a removable medium, such as a CD-ROM in a CD-ROM drive; etc. The memory may be read-only; write-only; both readable and writable; etc. The memory may be read-once; read-many; write-once; write-many; etc. The memory may be accessible in any suitable fashion, such as randomly; sequentially; either randomly or sequentially; etc. The memory may be dedicated to a particular computer or device; may be simultaneously connected to and shared by multiple computers or devices; may be shared over a network; etc. The memory may store the data in any computer-accessible medium, such as electronic, magnetic, optical, print, etc. The memory may be used to store data for a single task or application; may be used to store data for many tasks and applications; etc. The implementation of the computer-operable component is not important, so long as the computer-operable component is capable of storing and/or retrieving data.

“OASIS” is suggested to mean the Outcome and Assessment Information Set maintained by the U.S. Department of Health and Human Services, and particularly by the U.S. Centers for Medicare & Medicaid Services.

“Score” is suggested to mean a value indicator, where a value represented by one score has more or less value than a value represented by another score. “Score” may be numeric, such as a decimal number, and such numbers usually (but need not always) include a relative ordering of value, such as higher numbers denoting greater value than lower numbers, or such as higher numbers denoting less value than lower numbers. “Score” may also be non-numeric, such as in a letter grade system, where alphabetically earlier letters (“A”) imply more value than alphabetically later letters (“C”).

“Time point” is suggested to mean a period of time, such as a period of time during which an individual is represented as having a health state. The period of time may be short, such as a moment or an office visit, or long, such as a day or a month. The time point may describe a period within or around a described moment, or between two described moments.

### DETAILED DESCRIPTION

One or more aspects of the present disclosure are described with reference to the drawings, wherein like reference numerals are generally utilized to refer to like elements throughout, and wherein the various structures are not necessarily drawn to scale. In the following description, for purposes of explanation, numerous specific details are set forth in order to provide a thorough understanding of one or more aspects of the present disclosure. It may be evident, however, to one skilled in the art that one or more aspects of the present disclosure may be practiced with a lesser degree of these specific details. In other instances, well-known structures and devices are shown in block diagram form in order to facilitate describing one or more aspects of the present disclosure.

As noted hereinabove, this disclosure relates to techniques for automated, correlational health diagnosis, where a set of health indicators describing the health state of an individual (e.g., sweating, dizziness, and confusion) are correlationally linked with a set of conditions (e.g., diabetic hypoglycemic episode, stroke, and myocardial infarction) to compute the most likely condition (e.g., a likely diagnosis of a diabetic hypoglycemic episode.) The correlational likelihoods between health indicators and health diagnoses are stored in a diagnostic database, which can be queried with the individual's health indicators to determine the health diagnoses having the greatest correlation with the individual's health state. In addition to outputting the most likely health diagnoses, one or more health treatments may be recommended for addressing each likely health diagnosis. The information generated through the use of these techniques may also be sent to a health-related information system, such as a health information hierarchical classification system, for organized storage.

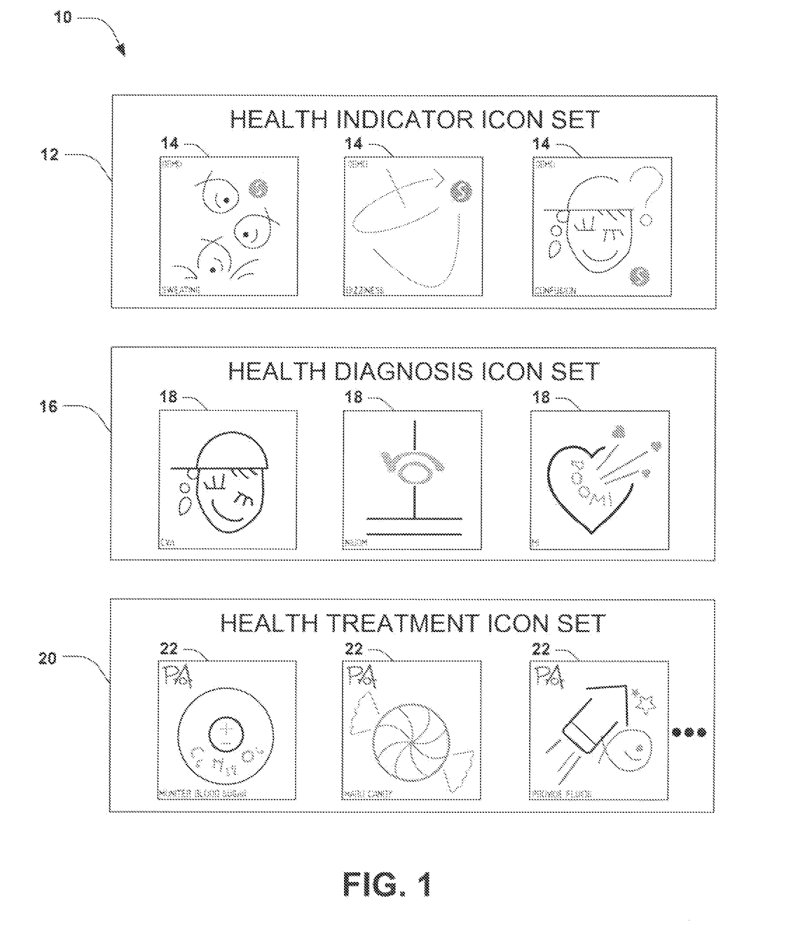

Many aspects of these techniques involve communication with users. It may be advantageous to communicate with users by displaying (and allowing the user to select as a form of input) icons of predominantly pictorial form that visually represent health-related concepts. The inclusion of icons of this form with such techniques may permit embodiments thereof to present health-related information to users in an easily cognizable, language- and literacy-independent manner, and to standardize the communication of health-related information among caregivers and the individual. FIG. 1 illustrates a portion of an exemplary icon set that may be used with a health diagnosis system, where the icon set 10 may comprise a health indicator icon set 12, where each health indicator icon 14 visually represents a health indicator of an individual, e.g., sweating, dizziness, and confusion. The exemplary icon set 10 may also include a health diagnosis icon set 16, where each health diagnosis icon 18 visually represents a health diagnosis that may describe the health state of an individual, e.g., a diabetic hypoglycemic episode, cerebral vascular accident, and myocardial infarction. The exemplary icon set 10 may also include a health treatment icon set 20, where each health treatment icon 22 visually represents a health treatment that may be performed to address a health diagnosis, e.g., a plan of action (PoA) item involving taking blood sugar levels via a glucometer, and if such blood sugar levels are abnormal, subsequent plan of action (PoA) items to administer candy if a low blood sugar level is again noted. A fail-safe may be included where all relevant plan of action (PoA) items are presented and exhausted until health indicators are either resolved or stabilized upon the conclusion of a visit.

The formation of a comprehensive and standardized icon set of predominantly pictorial form may serve to unify the communication of health-related information among various users of embodiments of these techniques, and may further be used in other systems (e.g., a health information hierarchical classification system that provides an electronic medical record of the health state of an individual) to produce a variety of intercommunicating health-related information systems that communicate with users in a common manner. While the icons presented in FIG. 1 are used in other figures provided herein, it will be appreciated that many icon sets may be devised that similarly represent health information, and that may be used in the manner described herein.

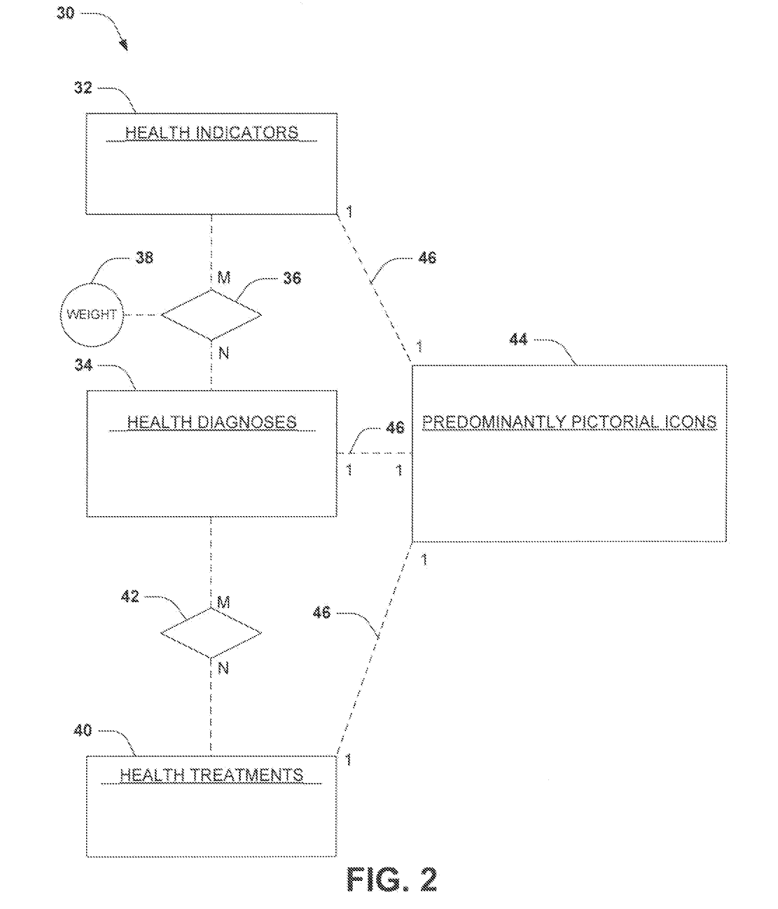

The techniques discussed herein utilize a diagnostic database that comprises a set of correlationally weighted relationships between health indicators and health diagnoses. This diagnostic database may be implemented in many ways, and one exemplary implementation is illustrated in FIG. 2 as a database schema in Entity-Relationship Diagram (ERD) form. It will be appreciated that the related entities in FIG. 2 are shown as having relationships with various constraints, such as “1:1” relationships, “1:N” relationships, and “M:N” relationships, and will be understood according to the conventions of Entity-Relationship Diagrams used to illustrate relational database schemas.

This exemplary diagnostic database schema 30 comprises one or more health indicators 32 and one or more health diagnoses 34, and a many-to-many (M:N) relationship 36 between the health indicators 32 and the health diagnoses 34. The structure of this relationship 36 permits the representation of a health diagnosis 34 (e.g., diabetic hypoglycemic episode, cerebral vascular accident, and myocardial infarction) with one or more health indicators 32 (e.g., sweating, dizziness, and confusion), and also permits each heath indicator 32 (e.g., sweating) to be indicative of one or more health diagnoses 34 (e.g., myocardial infarction and a cerebral vascular accident.) Each health indicator/health diagnosis relationship 36 has a diagnostic weight 38, which indicates the magnitude of the correlation between the health indicator 32 and the health diagnosis 34. For example, a health indicator representing dizziness may be highly correlated with a health diagnosis representing a cerebral vascular accident, therefore having a higher correlational weight attributed to the relationship, than with a health diagnosis representing acute myocardial infarction, which may have a lower correlational weight attributed to the relationship. For a health indicator that is not correlated with a health diagnosis (e.g., confusion as an indicator of a myocardial infarction), the correlational weight of the relationship may be represented as zero. For a health indicator that is negatively correlated with a health diagnosis (e.g., deep breathing as an indicator of choking), the correlational weight of the relationship may be represented as a negative value.

The exemplary diagnostic database schema 30 illustrated in FIG. 2 also comprises a relationship between health diagnoses 34 and health treatments 40, where each related health treatment 40 is intended to address the health diagnosis 34. For example, a health diagnosis 34 representing a diabetic hypoglycemic episode may be related to a health treatment 40 for ad ministering candy. The relationship 42 between the health diagnoses 34 and the health treatments 40 is illustrated as a many-to-many (M:N) relationship, such that each health diagnosis 34 may be related to more than one health treatment 40, and one health treatment 40 may be remedial for multiple health diagnoses 34.

The exemplary diagnostic database schema 30 illustrated in FIG. 2 also features a set of predominantly pictorial icons 46 that is related to the health indicators 32, health diagnoses 34, and health treatments 40. The predominantly pictorial icons 44 are related to each of these database items in a one-to-one (1:1) relationship 46, such that each icon 44 represents only one health indicator 32, health diagnosis 34, or health treatment 40, and such that each such item 32, 34, 40 is represented by only one icon 44.

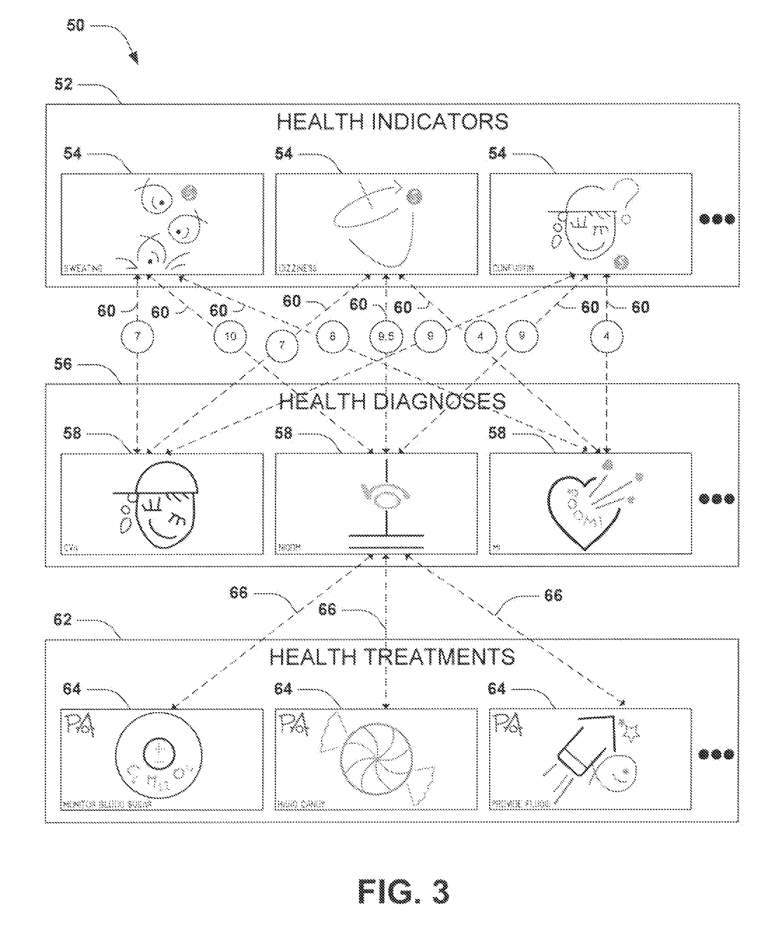

An exemplary diagnostic database formed in accordance with the diagnostic database schema 30 illustrated in FIG. 2 is presented in FIG. 3, in which a portion of the diagnostic database 50 comprising a set of three health indicators 52, a set of three health diagnoses 56 related to the health indicators set 52 (and where each such relationship comprises a correlational diagnostic weight), and a set of health treatments 62 related to the health diagnoses set 56. Each health indicator 54 in the heath indicator set 52 has a relationship 60 with each health diagnosis 58 in the health diagnosis set 56, where each such relationship 60 includes a correlational weight. Also, each health diagnosis 58 in the health diagnosis set 56 is related to at least one health treatment 64 in the health treatment set 62 that may be recommended for addressing each likely health diagnosis. The exemplary diagnostic database 50 is illustrated as having an icon associated with each health indicator 54, each health diagnosis 58, and each health treatment 64.

The diagnostic database utilized by the techniques described herein may be configured with many different structures. The data may be stored and managed by a relational database management system, such as Microsoft SQL Server, MySQL, or Oracle RDBMS. Alternatively, the data may be stored in a structured database file, such as Microsoft Access. The data comprising the database may also be stored in other formats (e.g., as one or more flat files), but may be logically accessed as if stored as a structured database file. The database may also be stored and accessed in many different forms, such as on a local storage device, on a network server accessed over a local network, or on a remote server accessed over the Internet. The data may also be structured in many convenient formats. For example, the data representations of the predominantly pictorial icons associated with the health indicators, health diagnoses, and health treatments may be stored within the database, or may be stored as part of a file system with database references (e.g., filenames) to the stored files. Many such configurations of the diagnostic database may be devised by those of ordinary skill of the art and used in accordance with the techniques described herein.

The data comprising the diagnostic database may also be generated in many ways. As one example, the data (e.g., the health indicators, the health diagnoses, and the weighted relationships therebetween) may be manually created based on the expertise and judgments of healthcare professionals. The data may also be devised through data mining of healthcare records to formulate correlations between recorded health indicators and resulting diagnoses by healthcare professionals in either a supervised or an unsupervised manner. The data may be stored as a static database, or may be updated over time to refine the data for improved diagnostic accuracy (again, with or without supervision by healthcare professionals.) Those of ordinary skill in the art may be able to devise many ways of filling the diagnostic database with data for use with the techniques described herein.

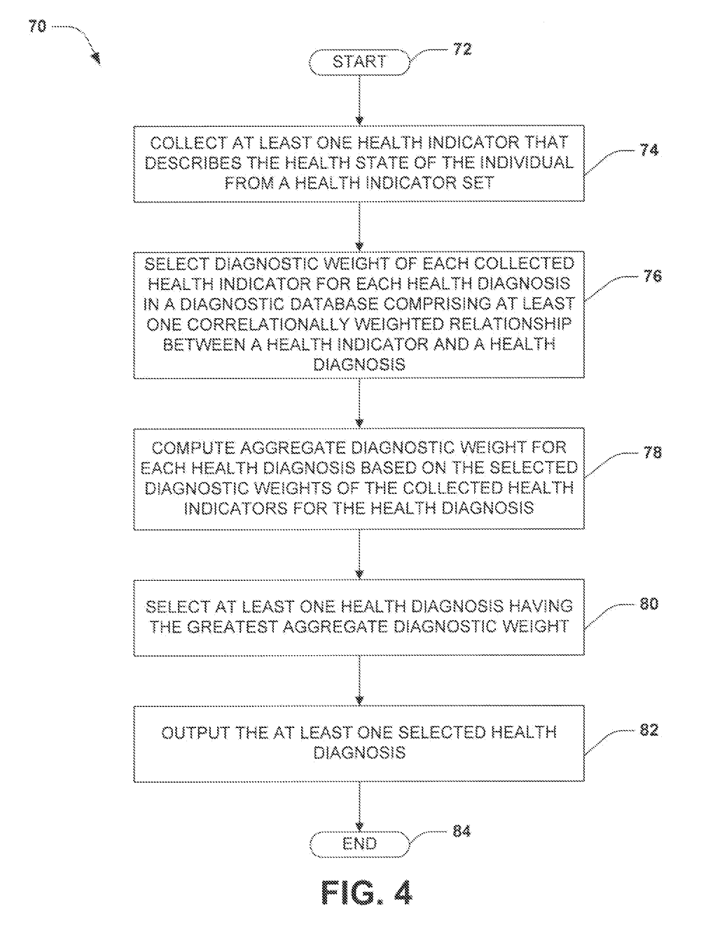

Having described the use of the icons in conjunction with these techniques and the diagnostic database for use herewith, this disclosure will now describe the techniques for performing automated correlational health diagnosis. One such technique is presented as the exemplary method of FIG. 4 for determining a health diagnosis of a health state of an individual. In FIG. 4, the method 70 begins at 72 and involves collecting at least one health indicator that describes the health state of the individual from a health set indicator 74. The exemplary method 70 also comprises selecting the diagnostic weight of each collected health indicator for each health diagnosis in a diagnostic database comprising at least one correlationally weighted relationship between a health indicator and a health diagnosis 76. For example, the method may query the diagnostic database to fetch the correlational weights of each relationship between the collected health indicators and the health diagnoses. The exemplary method 70 also comprises computing an aggregate diagnostic weight for each health diagnosis, based on the selected diagnostic weights of the collected health indicators for the health diagnosis 78. The selection of correlational weights 76 and computation of the aggregate diagnostic weight for each health diagnosis 78 provide a set of health diagnoses correlationally scored against the health indicators for the individual. The health diagnoses may therefore be ordered according to aggregate diagnostic weight, i.e., the correlational likelihood that each health diagnosis describes the individual's health state. Accordingly, the exemplary method 70 comprises selecting at least one health diagnosis having the greatest aggregate diagnostic weight 80, and outputting the at least one selected health diagnosis 82. By computing and outputting the most likely health diagnoses in this manner, the exemplary computer-implemented method 70 yields a health diagnosis for the individual, and the exemplary method 70 ends at 84.

Optionally, the method 70 may select one or more health treatments recommended for addressing each selected health diagnosis. Such health treatments may be included in the diagnostic database, and may be retrieved therefrom once one or more health diagnoses have been selected. The selected health treatments may be then be included in the diagnostic output of the method 70, thereby recommending a course of action for addressing the health diagnoses pertaining to the health state of the individual.

It will be appreciated that although the elements of the exemplary method 70 are discussed in the sequence presented in FIG. 4, the elements need not be performed in this order in order to utilize the techniques described herein. As one example, the method may collect and assess the health indicators serially, and may continue to refine the diagnosis as additional health indicators are received. Thus, upon collecting the first health indicator 74, the method selects the diagnostic weights for the first health indicator 76, computes the aggregate diagnostic weights of the health diagnoses as related with the first heath indicator 78, and selects the health diagnoses having the greatest aggregate diagnostic weights 80. The method may then collect a second health indicator 74 and perform the same processing 76, 78, 80 to produce a more accurate diagnosis, and this processing flow may be continued until all health indicators are collected. As another example, the selection of diagnostic weights from the diagnostic database 76 and the computing of the aggregate diagnostic weights 78 may be performed either simultaneously for each health indicator or in sequence.

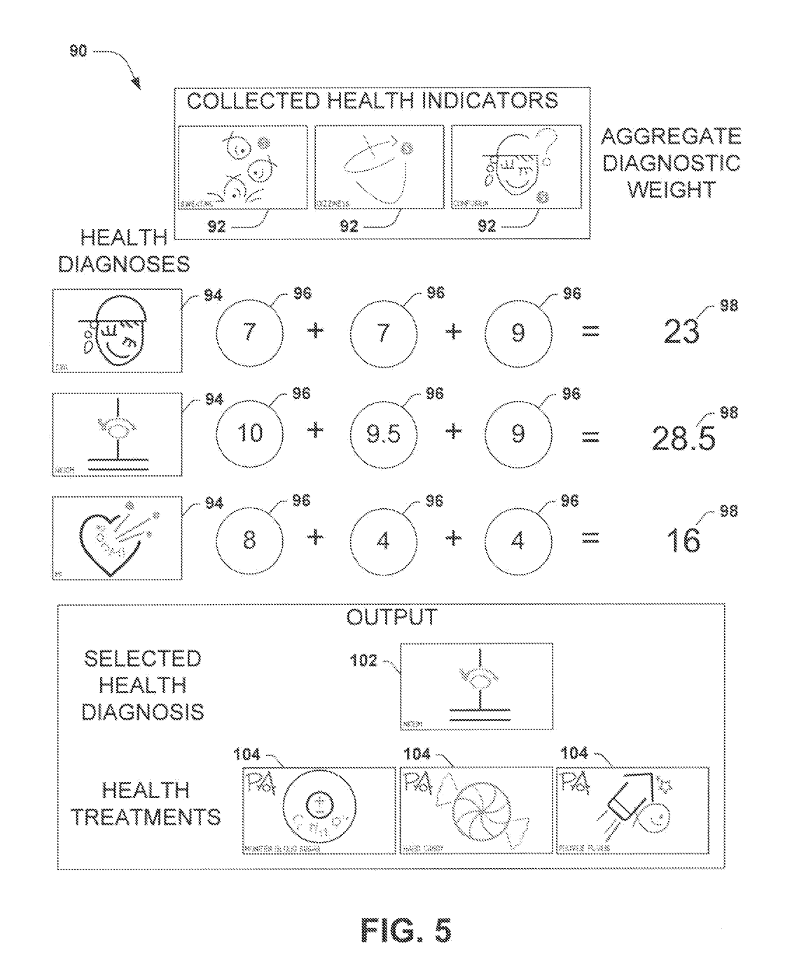

FIG. 5 illustrates the use of the portion of the exemplary diagnostic database 70 of FIG. 4 to produce an automated, correlational diagnosis based on a set of collected health indicators. In this illustrated use 90, the collected health indicators 92 describe an individual presenting with the symptoms of sweating, dizziness, and confusion. For each health diagnosis 94 in the diagnostic database 70 of FIG. 4, the diagnostic weights 96 are selected from the diagnostic database 70 for each of the collected health indicators 92. For example, the diagnostic database contains the health diagnosis “stroke,” so the exemplary use 90 involves selecting the diagnostic weights 96 of the relationships between “stroke” and “sweating,” “stroke” and “dizziness,” and “stroke” and “confusion.” Once the diagnostic weights 96 are selected for each health diagnosis 94 in the diagnostic database 70, the diagnostic weights 96 are aggregated to produce an aggregate diagnostic weight 98 for each health diagnosis 94. In the exemplary diagnostic database 70 of FIG. 4, the diagnostic weights 96 are numeric, and in this exemplary use 90, the diagnostic weights 96 are aggregated by summing the diagnostic weights 96 for each health diagnosis 94. For example, the diagnostic weights for “stroke” that have been selected (7 for “sweating,” 7 for “dizziness,” and 9 for “confusion”) are summed to produce an aggregate diagnostic weight 98 of 23. The aggregate diagnostic weights 98 are used to order to health diagnoses 94 by order of correlational diagnostic weight, and the health diagnosis 94 having the greatest aggregate diagnostic weight 98 is selected, i.e., the health diagnosis of “diabetic hypoglycemic episode.”

The result of this exemplary use 90 of the techniques disclosed herein is the (at least one) selected health diagnosis 102. Additionally, one or more health treatments 104 may also be selected from the diagnostic database 70 of FIG. 4 and may be recommended for addressing each likely health diagnosis. In this exemplary use 90, the selected health diagnosis of “a diabetic hypoglycemic episode” 102 is provided as output, along with health treatments 104 that may be effective for addressing the health state of the individual (e.g., providing hard candy, monitoring the individual's blood sugar, and providing fluids.)

The exemplary computer-implemented method 70 of FIG. 4 may be embodied in many ways in accordance with the techniques discussed herein. Some various embodiments will now be presented having various features and advantages.

The techniques discussed herein involve collecting a set of health indicators that describe the health state of the individual. This collecting may be performed in many ways. As one example, health diagnoses may be presented to a user of the method (e.g., the individual or a healthcare provider), such as by displaying icons of predominantly pictorial form that represent health indicators. The user may then select one or more health indicators, such as by touching icons on a touchscreen display, pointing at icons with a pointing device such as a mouse or stylus, or providing input via a keyboard. As another example, this collecting may comprise communicating with one or more sensors configured to detect one or more health indicators from an individual, such as a heart monitor configured to measure the heart rate of the individual and to generate health indicators related thereto (e.g., “rapid pulse.”)

The techniques discussed herein also involve selecting health diagnoses based on the aggregate diagnostic weights. The selecting may be performed by using the health diagnoses and aggregate diagnostic weights in many ways. As one example, the health diagnosis having the greatest diagnostic weight may be selected as output; in the case of multiple health diagnoses having the same greatest aggregate diagnostic weight, all such health diagnoses may be selected, or one may be preferentially designated for selection. As another example, the top several health diagnoses may be selected, and may be presented to the user in order of diagnostic weight, in order to notify the user of additional health diagnoses that may apply to the individual's health state. As still another example, all health diagnoses having an aggregate diagnostic weight above a minimum threshold of correlational likelihood may be selected and provided. As another variation, the diagnostic weights may be represented in the diagnostic database and aggregated in many ways. As one example, the diagnostic weights may be numeric values, e.g., correlational percentages or arbitrary scores, and may be aggregated according to a formula, e.g., summing the diagnostic weights. As another example, the diagnostic weights may be non-numeric identifiers, e.g., letter grades from “A” through “E” indicating the strength of the correlational relationship, and may be aggregated by choosing the average non-numeric identifier for each correlational relationship, e.g., by aggregating diagnostic weights “A”, “C”, “D”, and “D” into the aggregate diagnostic weight “C”. Many variations on the ordering of the elements of the exemplary method 70 of FIG. 4 may be devised by those of ordinary skill in the art and in accordance with the techniques presented herein.

When at least one health diagnosis has been selected, the selected health diagnoses are provided as output. The output may be provided in many ways. As one example, the output may be displayed for the user (including the individual and healthcare providers) to indicate the selected diagnoses, e.g., by displaying an icon of predominantly pictorial form for each selected diagnosis. The output may also be printed, such as by printing icons of predominantly pictorial form, and/or audibly represented, such as by generating speech indicating the health diagnoses. The output may also be used by the computer system in which the method is implemented. As one example, the output may be sent to one or more devices configured to provide healthcare functions to the individual. One such device that may advantageously utilize the health diagnostic output is a medication reminder device, such as disclosed in U.S. patent application Ser. No. 11/712,376 (“Device for Facilitating Compliance With Medication Regimen”), the entirety of which (except the claims) is incorporated herein by reference. A device of this type may utilize the health diagnostic output of the method, as well as the health treatment output of the method (e.g., instructions that comprise medication instructions.) As another example, the output may be stored as part of the individual's electronic medical record. One electronic medical record system in which the health diagnostic output of the method may be advantageously utilized is a health information classification scheme. One such health information scheme is disclosed in U.S. patent application Ser. No. 11/753,306 (“Health Information Hierarchical Classification Scheme and Methods and Systems Related Thereto”), the entirety of which (except the claims) is incorporated herein by reference. Another such health information scheme is the Outcomes and Assessments Information Set (OASIS) classification scheme created and utilized by the U.S. Department of Health & Human Services. Health information schemes of this type may be readily compatible with the health indicators, health diagnoses, and health treatments that together describe the health state of the individual, and so may incorporate the output into the individual's electronic medical record with little data translation and high accuracy. Many uses of the output of these techniques may be devised by those of ordinary skill in the art.

The techniques discussed herein may be embodied in many forms, each of which may include the various aspects described herein. As one example, the exemplary method 70 of FIG. 4 may be implemented on a computer and connected to a diagnostic database formed and managed in the manner suggested hereinabove. The computer implementation of this method may enable the application of these techniques to practical uses, such as the exemplary automated diagnosis 90 of FIG. 5.

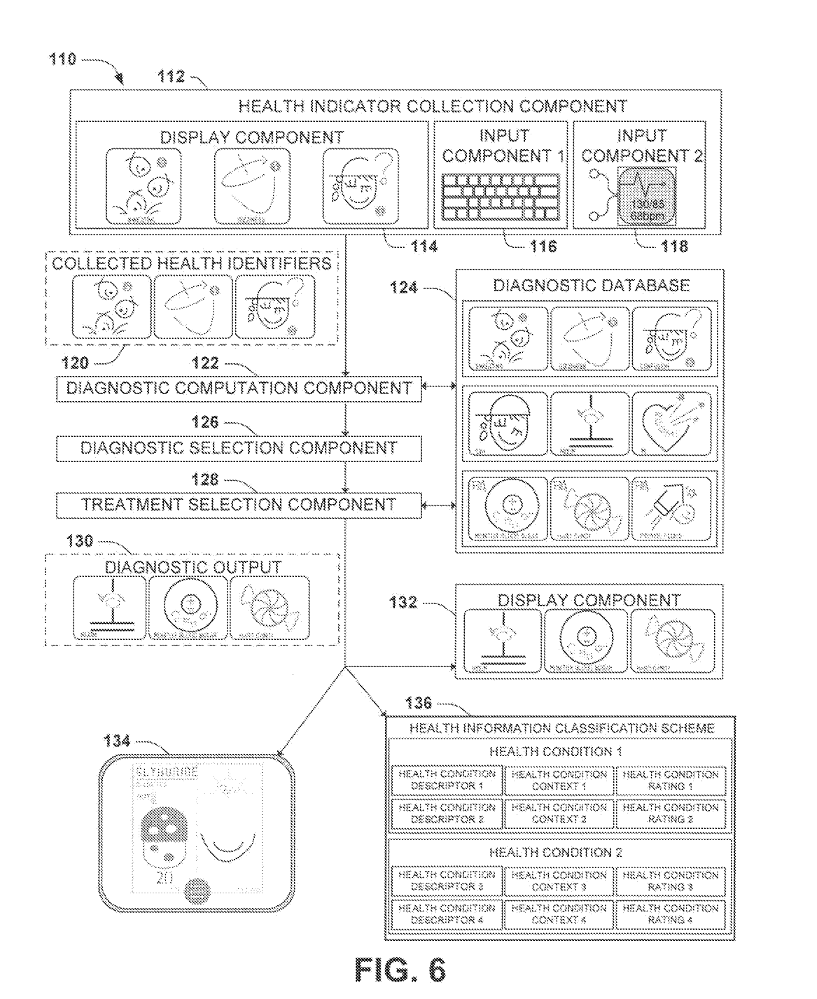

The techniques described herein may also be embodied in a system, comprising a health indicator collection component, a diagnostic database, a diagnostic computation component, a diagnostic selection component, and a health diagnosis output component. An exemplary system embodying these techniques is illustrated in FIG. 6, which depicts the integration of the components described above, along with some optionally additional components. (The exemplary system 110 of FIG. 6 is illustrated as processing the same information in the exemplary use 90 of FIG. 5.)

In FIG. 6, the system 110 comprises a health indicator collection component 112 configured to collect at least one health indicator that describes the health state of the individual. The health indicator collection component may comprise (e.g.) a display component 114, e.g. an LCD panel, configured to display icons of predominantly pictorial form representing the health indicators in the diagnostic database, and an input component configured to accept a user selection of at least one icon. A display component of this type may comprise many forms of user interface hardware for selecting the icons, such as (e.g.) a touchscreen interface, a pointing device such as a mouse or stylus, a keyboard 116, a microphone and a speech recognition package, etc. Alternatively or additionally, the health indicator collection component may comprise a communications device configured to communicate with at least one sensor configured to detect at least one health indicator relating to the health state of the individual. As one example, included in the exemplary system 110 of FIG. 6, a heart monitor 118 may generate health indicators related to the cardiac health of the individual, and may send the health indicators to the health indicator collection component 112. Multiple components may comprise the health indicator collection component for generating multiple kinds of health indicators as input from various sources.

The health indicator collection component 112 yields a set of collected health indicators 120, e.g., health indicators for “sweating,” “dizziness,” and “confusion.” The system 110 further comprises a diagnostic computation component 122 configured to receive the collected health identifiers 120 from the health indicator collection component 112 and to interact with a diagnostic database 124 comprising at least one diagnostically weighted relationship between a health indicator and a health diagnosis, which may be formed as described herein. The diagnostic computation component 122 is configured to select from the diagnostic database 124 the diagnostic weight of each collected health indicator 120 as related to each health diagnosis in the diagnostic database 124, and to compute an aggregate diagnostic weight for each health diagnosis based on the selected diagnostic weights of the collected health indicators 120 for the health diagnosis.

The diagnostic computation component 122 thereby produces a set of health diagnoses ordered by aggregate diagnostic weights with respect to the health indicators for the individual. The system 110 further comprises a diagnostic selection component 126 configured to receive the health diagnoses and aggregate diagnostic weights from the diagnostic computation component 122 and to select at least one health diagnosis having the greatest aggregate diagnostic weight for the health state of the individual computed by the diagnostic computation component.

The system 110 may comprise a treatment selection component 128 configured to select at least one health treatment related to the at least one health diagnosis selected by the diagnostic selection component 126. The treatment selection component 128 may interact with the diagnostic database 124 to retrieve the health treatments related to each selected health diagnosis, and may include the selected health treatments with the selected health diagnoses as diagnostic output.

The diagnostic selection component 126 thereby produces a set of selected health diagnoses, and may include one or more health treatments selected by the treatment selection component 128. The health diagnoses and (optionally) the related health treatments comprise the diagnostic output 128 of the diagnostic selection component. The system 110 further comprises a health diagnosis output component configured to output the at least one selected health diagnosis (and, if included, the one or more health treatments for each health diagnosis.) The health diagnosis output component may comprise, e.g., a display component 130 configured to display at least one icon of predominantly pictorial form representing the at least one selected health diagnosis, and may further display at least one icon of predominantly pictorial form representing the related health treatments for each health diagnoses. Alternatively or additionally, the health diagnosis output component may comprise a communications device configured to transmit a health treatment comprising a medication instruction to a medication regimen compliance device, such as the “Device for Facilitating Compliance With Medication Regimen” referenced hereinabove. A medication reminder device of this type 134 is included in the exemplary system 110 of FIG. 6. Alternatively or additionally, the health diagnosis output component may also transmit the diagnostic output 130 to an electronic medical record system, such as a system implementing the “Health Information Hierarchical Classification Scheme” referenced hereinabove, for inclusion in the electronic medical record of the individual. A health information system of this type 136 is included in the exemplary system 110 of FIG. 6.

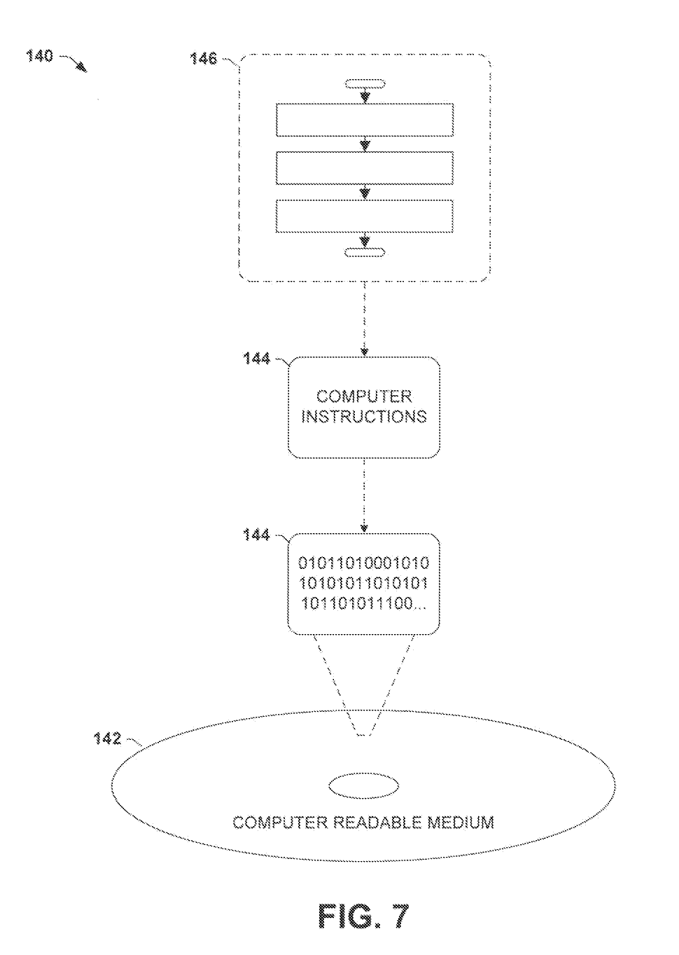

The techniques disclosed herein may also be embodied in other forms. As one example, these techniques may be implemented as a computer-readable medium comprising processor-executable instructions configured to implement the methods and/or systems disclosed herein. One example of this form of embodiment is illustrated in FIG. 7, wherein the implementation 140 comprises a computer-readable medium 142 (e.g., a CD-R, DVD-R, or a platter of a hard disk drive), on which is encoded computer-readable data 144. This computer-readable data 144 in turn comprises a set of computer instructions 146 that operate in accordance with this disclosure. For example, and as illustrated in FIG. 7, the computer instructions 146 may implement a method 148 in accordance with this disclosure, such as the method illustrated in FIG. 4. As another example, these techniques may be embodied as a carrier wave containing processor-executable instructions configured to implement the methods and/or systems disclosed herein. The processor-executable instructions embedded in such a computer medium and/or carrier wave may include at least one data representation of an icon of predominantly pictorial form representing one of a health indicator, a health diagnosis, and a health treatment.

Although the disclosure has been shown and described with respect to one or more implementations, equivalent alterations and modifications will occur to others skilled in the art based upon a reading and understanding of this specification and the annexed drawings. The disclosure includes all such modifications and alterations and is limited only by the scope of the following claims. In particular regard to the various functions performed by the above described components (assemblies, elements, devices, circuits, etc.), the terms (including a reference to a “means”) used to describe such components are intended to correspond, unless otherwise indicated, to any component which performs the specified function of the described component (i.e., that is functionally equivalent), even though not structurally equivalent to the disclosed structure which performs the function in the herein illustrated exemplary implementations of the disclosure. In addition, while a particular feature of the disclosure may have been disclosed with respect to only one of several implementations, such feature may be combined with one or more other features of the other implementations as may be desired and advantageous for any given or particular application. Furthermore, to the extent that the terms “includes” “having”, “has”, “with”, or variants thereof are used in either the detailed description or the claims, such terms are intended to be inclusive in a manner similar to the term “comprising.” Also, “exemplary” as utilized herein merely means an example, rather than the best.

1. A computer-implemented method of determining a health diagnosis for a health state of an individual, comprising:
collecting at least one health indicator represented in a diagnostic database that describes the health state of the individual by;
displaying icons of predominantly pictorial form representing the health indicators in the diagnostic database, and
upon user selection of at least one icon, collecting the health indicator represented by the at least one selected icon;
selecting the diagnostic weight of each collected health indicator for each health diagnosis in the diagnostic database comprising at least one correlationally weighted relationship between a health indicator and a health diagnosis; and
computing an aggregate diagnostic weight for each health diagnosis based on the selected diagnostic weights of the collected health indicators for the health diagnosis;
selecting at least one health diagnosis having the greatest aggregate diagnostic weight; and
outputting the at least one selected health diagnosis by displaying at least one icon of predominantly pictorial form respectively representing the at least one selected health diagnosis.

2. The computer-implemented method of claim 1, where computing the aggregate diagnostic weight of the health diagnosis comprises summing the diagnostic weights of the collected health indicators related to the health diagnosis.

3. The computer-implemented method of claim 1, where collecting the at least one health indicator comprises:
displaying icons of predominantly pictorial form representing the health indicators in the diagnostic database, and
upon user selection of at least one icon, collecting the health indicator represented by the at least one selected icon.

4. The computer-implemented method of claim 1, where collecting the at least one health indicator comprises communicating with one or more sensors configured to detect one or more health indicators from an individual.

5. The computer-implemented method of claim 1, where outputting the at least one selected health diagnosis comprises displaying an icon of predominantly pictorial form representing the at least one selected health diagnosis.

6. The computer-implemented method of claim 1, wherein the diagnostic database comprises at least one relationship between each health diagnosis and at least one health treatment, and the method further comprising:
after selecting the at least one health diagnosis, selecting at least one health treatment from the diagnostic database that is related to the at least one selected health diagnosis; and
outputting the at least one selected health treatment for each of the at least one selected health diagnoses.

7. The computer-implemented method of claim 6, where outputting the at least one selected health treatment comprises displaying an icon of predominantly pictorial form representing the at least one selected health treatment.

8. The computer-implemented method of claim 6, where outputting the at least one selected health treatment comprises transmitting at least one data representation of a selected health treatment comprising a medication instruction to a medication regimen compliance device.

9. The computer-implemented method of claim 1, further comprising: relating the collected health indicators to at least one health condition in a health information classification scheme.

10. A computer-readable medium comprising processor-executable instructions configured to implement the method of claim 1.

11. The computer-readable medium of claim 10, the processor-executable instructions further comprising at least one data representation of an icon of predominantly pictorial form representing one of a health indicator, a health diagnosis, and a health treatment.

12. A computer data signal embodied in a carrier wave containing processor-executable instructions configured to implement the method of claim 1.

13. The computer data signal of claim 12, the processor-executable instructions further comprising at least one data representation of an icon of predominantly pictorial form representing one of a health indicator, a health diagnosis, and a health treatment.

14. A computer system for determining a health diagnosis for a health state of an individual, comprising:
a health indicator collection component configured to collect at least one health indicator that describes the health state of the individual;
a diagnostic database comprising at least one diagnostically weighted relationship between a health indicator and a health diagnosis, respective health indicators and respective health diagnoses associated with at least one icon of predominantly pictorial form representing the respective health indicators and the respective health diagnoses;
a diagnostic computation component configured to select from the diagnostic database the diagnostic weight of each collected health indicator as related to each health diagnosis in the diagnostic database and to compute an aggregate diagnostic weight for each health diagnosis based on the selected diagnostic weights of the collected health indicators for the health diagnosis;
a diagnostic selection component configured to select at least one health diagnosis having the greatest aggregate diagnostic weight for the health state of the individual computed by the diagnostic computation component; and
a health diagnosis output component comprising a display component configured to display at least one icon of predominantly pictorial form representing the at least one selected health diagnosis.

15. The computer system of claim 14, where the diagnostic computation component is configured to compute the aggregate diagnostic weight of the health diagnosis by summing the selected diagnostic weights of the collected health indicators related to the health diagnosis.

16. The computer system of claim 14, where the health indicator collection component comprises:
an input component configured to accept a user selection of at least one icon,
wherein the health indicator selection component is configured to collect the at least one health indicator represented by the at least one icon selected by the user through the input component.

17. The computer system of claim 16, where the health indicator collection component comprise a communications device configured to communicate with at least one sensor configured to detect at least one health indicator relating to the health state of the individual.

18. The computer system of claim 14, where the health diagnosis output component comprises a display component configured to display at least one icon of predominantly pictorial form representing the at least one selected health diagnosis.

19. The computer system of claim 14, where the diagnostic database comprises at least one relationship between each health diagnosis and at least one health treatment, and the system further comprises:
a treatment selection component configured to select at least one health treatment related to the at least one health diagnosis selected by the diagnostic selection component,
and where the health diagnosis output component is configured to output the at least one selected health treatment for each of the at least one selected health diagnoses.

20. The computer system of claim 19, where the health treatment output component comprises a display component configured to display at least one icon of predominantly pictorial form representing the at least one selected health treatment.

21. The computer system of claim 19, where the health treatment output component comprises a communications device configured to transmit a health treatment comprising a medication instruction to a medication regimen compliance device.

22. A computer-readable medium comprising processor-executable instructions configured to implement the system of claim 14.

23. The computer-readable medium of claim 22, the processor executable instructions further comprising at least one data representation of an icon of predominantly pictorial form representing one of a health indicator, a health diagnosis, and a health treatment.

24. A computer data signal embodied in a carrier wave containing processor-executable instructions configured to implement the system of claim 14.

25. The computer data signal of claim 24, the processor-executable instructions further comprising at least one data representation of an icon of predominantly pictorial form representing one of a health indicator, a health diagnosis, and a health treatment.
